(19) United States
(12) Patent Application Publication
(10) Pub. No.: US 2018/0247701 A1
(43) Pub. Date: Aug. 30, 2018

(54) System and Method to Combine, View, and Distribute Patient Health Records from Multiple Sources

(71) Applicant: Kirstan A. Vandersluis, Colorado Springs, CO (US)

(72) Inventor: Kirstan A. Vandersluis, Colorado Springs, CO (US)

(21) Appl. No.: 15/905,821

(22) Filed: Feb. 26, 2018

Related U.S. Application Data
(60) Provisional application No. 62/463,710, filed on Feb. 26, 2017.

(51) Int. Cl. G16H 10/60; G06F 17/30

(52) U.S. Cl. CPC G16H 10/60; G06F 17/30979

(57) ABSTRACT

A system and method is described that automatically collects health information from multiple health data provider sources, combines it into a single database, then provides a view of the information on a body map. The body map can be a drawing, photograph, or other visual model, and can be changed over time as the patient advances in age. While the image may change over time, the system continues to plot the information in the correct body location.

### CROSS-REFERENCE TO RELATED APPLICATIONS

This application claims the benefit of U.S. Provisional Application Ser. No. 62/463,710, filed Feb. 26, 2017, which is hereby incorporated by reference in its entirety.

### FIELD OF INVENTION

The present invention relates to combining, viewing, and distributing patient health records from multiple sources.

### BACKGROUND OF THE INVENTION

The present invention relates to computer software running on a device for multiple platforms including but not limited to web, mobile, wearable technologies, holograms, game systems, motion-capture, TV, and other devices that allows people to collect and manage medical and health records from multiple providers into a single database, with comprehensive views of health information designed for understandability for the patient as well as the provider.

Providers maintain records of patients in computer systems (electronic medical records-EMR systems) or on paper. The typical person in the US has 5 to 10 healthcare providers. A person will then have health records spread across 5-10 different systems. This severely limits the patient's ability to have a complete view and understanding of their health condition. A patient with full access to their health information has a higher likelihood of successful health outcomes.

When medical providers treat a patient, they first gather the patient's health history through a questionnaire and verbal interview process. If the patient does not fully or accurately recall their health history, the provider will make a diagnosis based on incomplete or inaccurate health history, affecting the quality and efficiency of the care provided. Access to a complete and accurate health history will assist the provider to deliver better and more efficient healthcare.

Lack of control over health information creates inertia in healthcare delivery from the patient's perspective. Patients feel tied into current providers, making it seem infeasible to find the best valued provider at any given time. Instead, the patient is highly likely to seek care from their current provider, even if they have to wait extended amounts of time for an appointment. Delays in care delivery reduce the likelihood of successful health outcomes.

From a provider's perspective, controlling records and limiting sharing provides a business benefit by making it less likely a patient will switch to a different provider. Since patients are apt to wait for an appointment with the holder of their records, other providers are much less likely to get care business from patients who feel locked in to their current provider. The result is that healthcare capacity is not efficiently used as patients are discouraged from shopping around for the best value for their healthcare dollars. Providers who have skills and available capacity are less likely to utilize that capacity, as prospective patients are unlikely to seek them out.

Patients who are better informed about their condition and care plan achieve better health outcomes. The key to achieving the benefit of a more informed patient is providing pertinent information at the time of care, in a form that the patient can review after the visit. Having access to care information after the visit enables the patient to review and confirm the care plan, achieving better conformance. Providing medical records and doctor notes to a patient for ongoing and permanent reference improves overall care.

What is needed is a system and method for patients to take control of and manage their health records to increase their understanding of their health condition and plans (leading to better outcomes), and to enable free-market economics of encouraging patients to act as “consumers” of healthcare, leading to better system-wide efficiency of care delivery. Patients can find the best available provider, given their health history, current condition, convenience of scheduling, and availability of providers.

### SUMMARY

The present invention comprises a system and method to configure a system to automatically collect health records from multiple providers, collect the data, then normalize the data into a common format for presentation, analysis, predictive diagnosis, and other processes, then store the data for long term (life long) use, avoiding the reliance on individual providers to store data (as these change from time to time, causing permanent loss of information).

The combined set of records are projected onto several views to aid in rapid viewing and understanding of a user's health. The views are intended for both patient and provider understanding of health history and current conditions to increase efficiency in health care delivery. Views allow filtering of data to enable the user to see information of interest. Dimensions of filtering include medical areas (orthopedics, dermatology, internal medicine, etc.), status of the condition (new, ongoing/chronic, recovered/corrected, etc.), time range, and others.

The combined health record is also intended to facilitate easy switching between providers, which is a catalyst for “consumer driven health care” where patients make a conscious decision on which provider to select based on judgment of value (a combination of price, convenience, expected outcome, and other factors). Historically, patients will wait in line to see their normal selected provider for a particular condition, since the provider has their full records. Now, when a patient has all their records at hand, many will not feel locked in to a single provider, and can thus select the provider of best value in their judgment.

### BRIEF DESCRIPTION OF THE DRAWINGS

For a more complete understanding of the present invention and its features and advantages, reference is now made to the following description, taken in conjunction with the accompanying drawings, in which:

FIG. 1 illustrates an exemplary system environment wherein the system for integrating and distributing health records operates.

FIG. 2 illustrates an exemplary health information management system.

FIG. 3 illustrates a database table named “user” to store information for the one or more instances of a user of the system.

FIG. 4 illustrates a database table named “accountManager” to store a relationship between users such that one user is given permission to view health information of another as in the case of a care giver providing care to a child or elderly parent.

FIG. 5 illustrates a database table named “provider” to store information about health record provider systems that contribute health records to a user's overall health information.

FIG. 6 illustrates a database table named “fetchJob” to store information about which health data provider system to connect to, along with credential information.

FIG. 7 illustrates a database table named “record” to store information about a health problem and its location on a visual body map.

FIG. 8 illustrates a computer system, an instance of which is used for each system as described in the following sections.

FIG. 9 is a picture of a computer screen rendered by an embodiment of the current invention.

FIG. 10 is a second picture of a computer screen rendered by an embodiment of the current invention, showing a realistic photograph of a user with superimposed health information.

FIG. 11 illustrates computer instructions to retrieve health information from a health data provider.

FIG. 12 illustrates a reference table where key words relating to the human body are translated to coordinates on a body map.

### DETAILED DESCRIPTION

The following detailed description refers to the accompanying drawings. Wherever possible, the same reference numbers are used in the drawings and the following description to refer to the same or similar parts. While several illustrative embodiments are described herein, one skilled in the art will be able to implement modifications, adaptations and other implementations. For example, substitutions, additions or modifications may be made to the components illustrated in the drawings, and the illustrative methods described herein may be modified by substituting, reordering, removing, or adding steps to the disclosed methods. Accordingly, the following detailed description should not be limited to the disclosed embodiments and examples. Instead, the proper scope is defined by the appended claims.

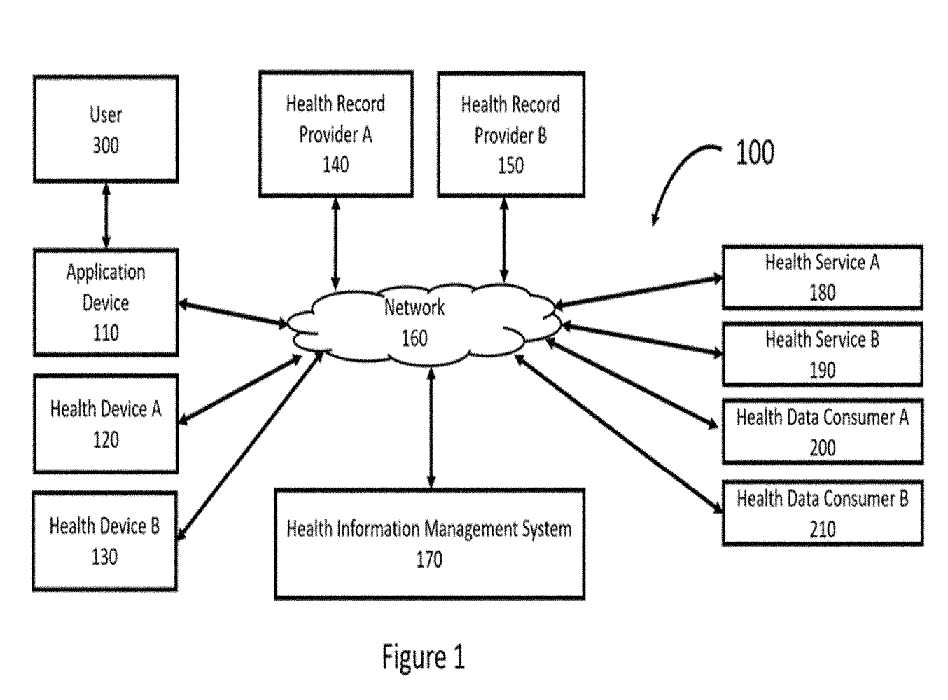

The present disclosure enables accessing health records from many sources in order to provide benefits to the patient, family, and caregivers. FIG. 1 shows an environment of systems 100, connected by a network 160, whereby systems can communicate with one another in a secure manner to allow health records to be accessed. The environment may comprise a network 160, an application device 110 which is a device that can interact with a human user 300, a system which manages electronic health records with a means to distribute information, for example health record provider A 140 and health record provider B 150, a health device A 120, health device B 130, a health information management system 170, a health service A 180, a health service B 190, a health data consumer A 200, and a health data consumer B 210.

In an exemplary environment of systems 100, in which embodiments consistent with the present disclosure may be practiced and implemented, the systems are able to communicate securely with one another, in a manner reflecting the security policies of each system. For example, the system 170 may be configured to receive data over an electronic network 160 (e.g., the Internet), from health record provider A 140, analyze the data, incorporate the data into a database, then transmit a merged or combined data set from the database to another system, such as application 110, which may in turn display the information to a user 300.

The following paragraphs describe the characteristics of example systems in the environment of systems 100 connected by a network 160 as shown in FIG. 1.

Application device 110 is a device such as a mobile phone or internet browser, which is capable of showing the health information transferred to it from the health information management system 170.

Health device A 120 and health device B 130 are health devices that can perform a health monitoring function such as measuring blood pressure, heart rate, or weight, and transmit that data over the network 110 to the health information management system 170.

Health record provider A 140 and health record provider B 150 are examples of systems that store health records associated with patients and make those records, or portions thereof, available to patients or their representatives through various technical means. An example of this type of system is an electronic health record (EHR) system in use by a health provider where a user 300 receives care, and providers enter information about the user 300. An EHR may make the information available via a patient portal, where a user 300 can log in, navigate to the appropriate page, and download the records in a predefined format. An EHR may alternatively make the data available through an application programming interface (API) using predefined interface mechanisms and formats.

A health service A 180 and health service B 190 are examples of systems that provide a service on behalf of the user 300 by examining the data transferred by the health information management system 170 on behalf of the user 300, performing some function such as a medical diagnosis, then returning the result to the health information management system 170 for later use of the user 300.

A health data consumer A 200 and health data consumer B 210 are examples of systems representing organizations that are interested in receiving and processing health data for their own purposes, for example a medical research organization or a population health organization. The health information management system 170 is capable of sending health information to these systems upon authorization by the user 300.

The health information management system 170 is a system that manages health information on behalf of a user 300. It collects information from other systems such as application device 110, health record provider A 140, health record provider B 150, health device A 120, health device B 130, health service A 180, and health service B 190, where the data is manipulated and stored for later use.

A user 300 of application 110 may be a patient, or may be a caregiver such as a parent in the context of caring for her child. A user 300 could be both a patient and caregiver, in which case it is beneficial for health information to be accessible for bot the patient and individuals for which the user provides care. The present disclosure allows the user to view and manage her own health information accessed from one or more sources, and to view and manage health information for one or more patients for which she is given authorization. The user can view the health information, and control the information which flows to presentation to patients, family, and caregivers, and how the newly combined data is useful for advanced purposes benefiting the patient and the broader healthcare industry.

All data is managed in a highly secure manner compliant with security standards such as NIST 800-53 and HIPAA compliant technology guidelines. Data is encrypted both at rest in the health information management system 170 and while in transit across the network 160.

Each system within the environment 100 is capable of managing data on behalf of a particular user 300, in a manner that ensures that data is appropriately associated with a specific user, so that data associated with a specific user is not unintentionally mixed together with data associated with other users.

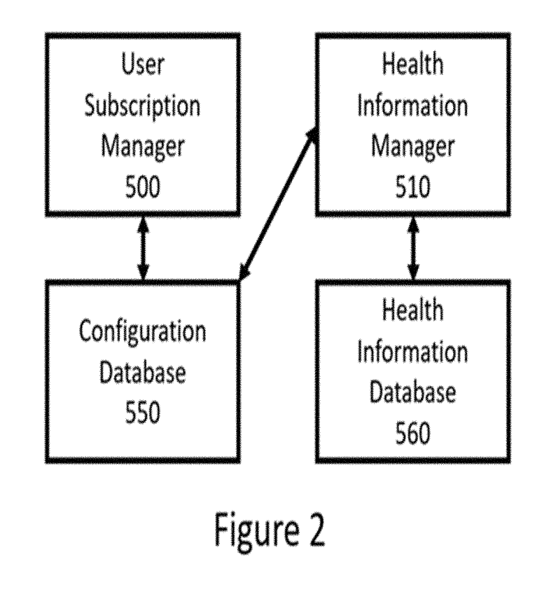

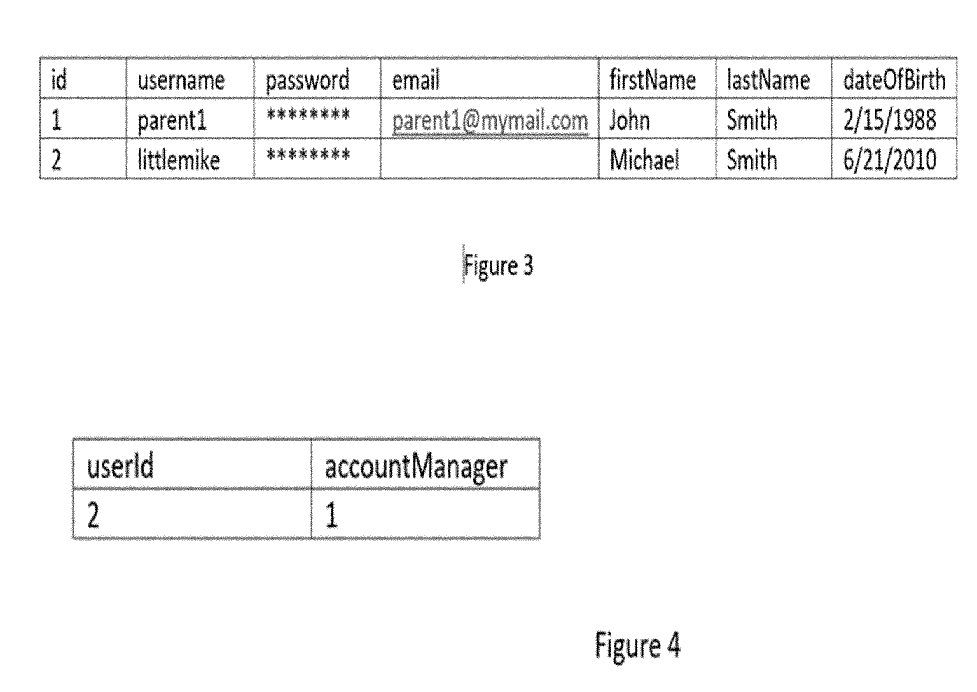

The present disclosure describes how a user 300 can subscribe to a service and provide instructions for collecting health information from multiple sources, and display the information on a single device. FIG. 2 illustrates an exemplary set of components which provide the capability for health information management system 170. A user subscription manager 500 enables a user 300 to subscribe to the services for the health information management system 170. In one embodiment of the disclosure, when a user 300 wishes to subscribe, the user subscription manager 500 collects a user name, password, email address, name, and other attributes as shown in FIG. 3, the “user” table. This information is stored and maintained in the configuration database 550. In this example, two users are shown, named John Smith and Michael Smith. One skilled in the art would collect this information from the user with a suitable interface, ensure the password follows adequate security guidelines, and the email address is valid. If one of the users is a child of another users, as could be the case in this example (note the same last name and dates of birth), the primary care giver would set up the account for the dependent. Note that the password field is shown as asterisks. A real password would be stored as a hash so that the password is not known by anybody except the user that created the account. An attempt to recover the password by a user would follow the standard conventions that a new password would need to be created. In this disclosure, password fields are always shown as asterisks. In the user table in FIG. 3, the “id” field is a unique identifier for a record. Username is a unique name identifying a user, email is the email address for a user. Other attributes include the users first name “firstName”, last or surname “lastName”, and date of birth “dateOfBirth”. The id field is a unique identifier for the user which will be used to relate other information in other tables, including health records, health data providers, and other information. The username and password field are credentials for a user 300 to log into the application device 110, and will be verified by the health information management system 170, after which the user 300 will be shown information from the health information management system 170.

To support a caregiver relationship, one user may manage the account of another. In that case, a relationship must be established and tracked to enforce this relationship. FIG. 4, table “accountManager” is configured to show a relationship that for the user with userId 2, which corresponds to the user record for Michael Smith in FIG. 3 user table, the user with userId 1 is an account manager. As user with ID 1 is John Smith in FIG. 3 user table, John Smith will be enabled to view all the information with the account for Michael Smith. This table is stored and maintained in the configuration database 550.

In a preferred embodiment of the invention, a configuration database 570 stores information about one or more health record providers such as health record provider A 140 and health record providers B 150, using a database table format as shown in FIG. 5, “provider” table. This table is managed by configuration database 570. Each record in this table identifies a health data provider which the system is capable of connecting to, and retrieving health information. The “id” attribute is a unique identifier for the record, the “name” attribute is a readable name representing the data source, for example the name of the practice with a patient portal that the system will connect to, the interfaceAddress attribute is an address, often a universal resource locator (URL), and the “script” attribute is the name of an executable script that is designed to connect to the data provider system, navigate as needed, and read or download related health information. In a preferred embodiment of the invention, the script field also enumerates the parameters needed by the script, such as the interfaceAddress, and credentials (username and password for example) for the user whose health information will be retrieved. For example, executing the script “/conf/providers/epf.js” and providing parameters “https://efp.com/portal”, “jsmith”, and “********” will result in these data values being passed at runtime to the script, where the script will log into a portal at that interface address using the username “jsmith” and supplied password, in order to read or download current health information to incorporate into the system. The provider table is stored and maintained in the configuration database 550. This database and table is configured to be accessible from

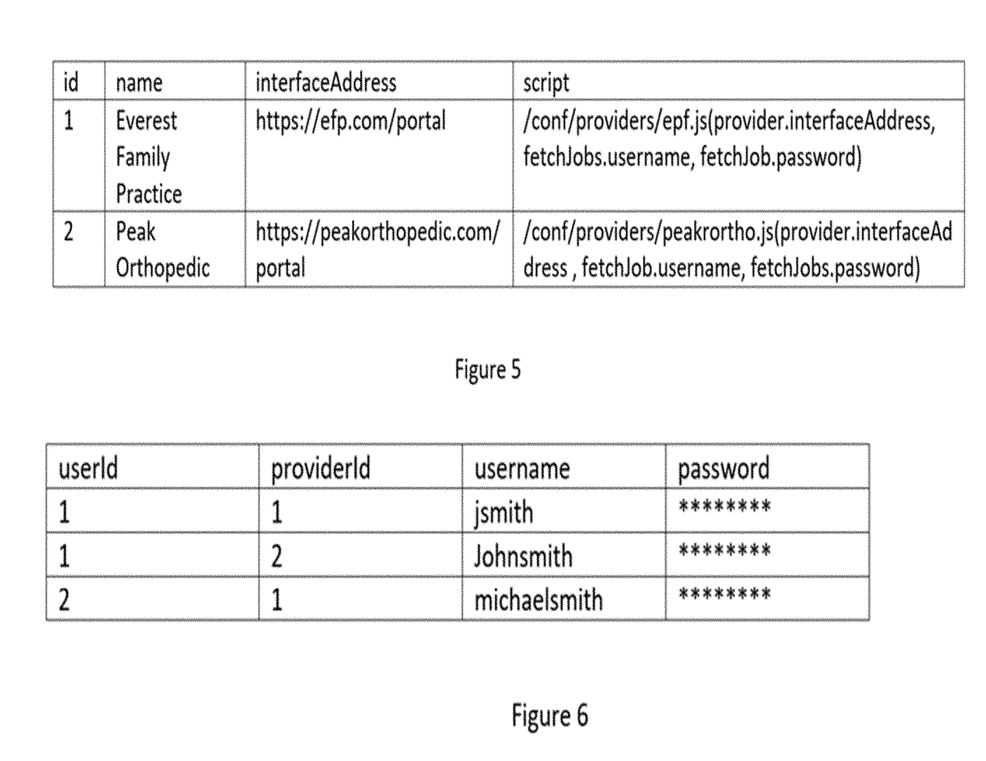

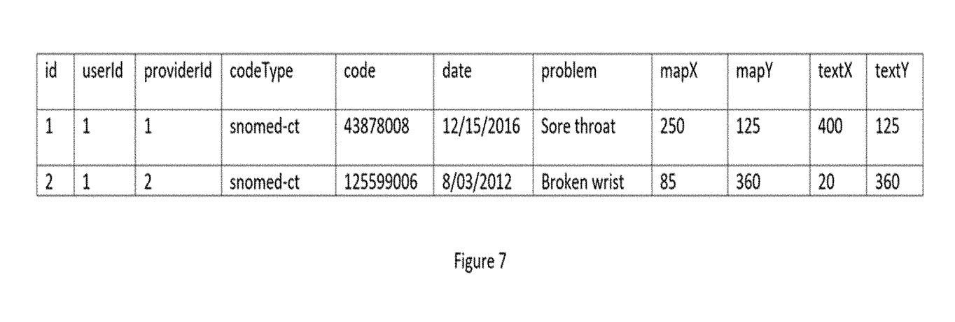

In a preferred embodiment of the invention, FIG. 6 shows a database table, “fetchJob”, which lists the health data providers to access, and the credentials needed to access each health data provider. In the example, the user represented by userId 1, has two records configured, each to access two different health data providers, including the health data provider with identifiers 1 and 2, which reference corresponding records in the “provider” table shown in FIG. 5. Linking the providerId to the corresponding ID in the provider table, the example indicates that the Everest Family Practice data provider will be accessed with username “jsmith” (first data record, FIG. 6), the Peak Orthopedic data provider will be accessed with username “johnsmith” (second data record, FIG. 6), and the Everest Family Practice data provider will be accessed with username “michaelsmith” (third data record, FIG. 6). The first two health data sets will be associated with user with identifier 1 “John Smith”, and the third with user with identifier 2 “Michael Smith”. The “fetchJob” table is stored and maintained in the configuration database 550.

In one embodiment of the invention, FIG. 6 shows a database table, “record”, which is a highly simplified definition of a health record, describing a condition, problem, procedure, or other aspect which is considered “health information”. In the example, two records are shown, both associated with the user with identifier 1 “John Smith” as indicated by the userId field. Each record contains information about a health problem, the health data provider form which it came (indicated by providerId field), a code representing the problem “code”, and the code system from which the code is defined (“codeType”), a description of the health problem or information item (“problem”), and location information which shows where on a 2 dimensional body map the problem occurs (mapX, mapY), and a location on the body map view where a textual description is placed (textX, textY). The codeType, code, date, and problem attributes are examples of data retrieved from a health data provider A 140 or health data provider B 150. The map and text location information is derived from this data, and references a coordinate system defined in terms of a canonical body map as will be described in a later section.

The “record” table is stored and maintained in the health information database 560.

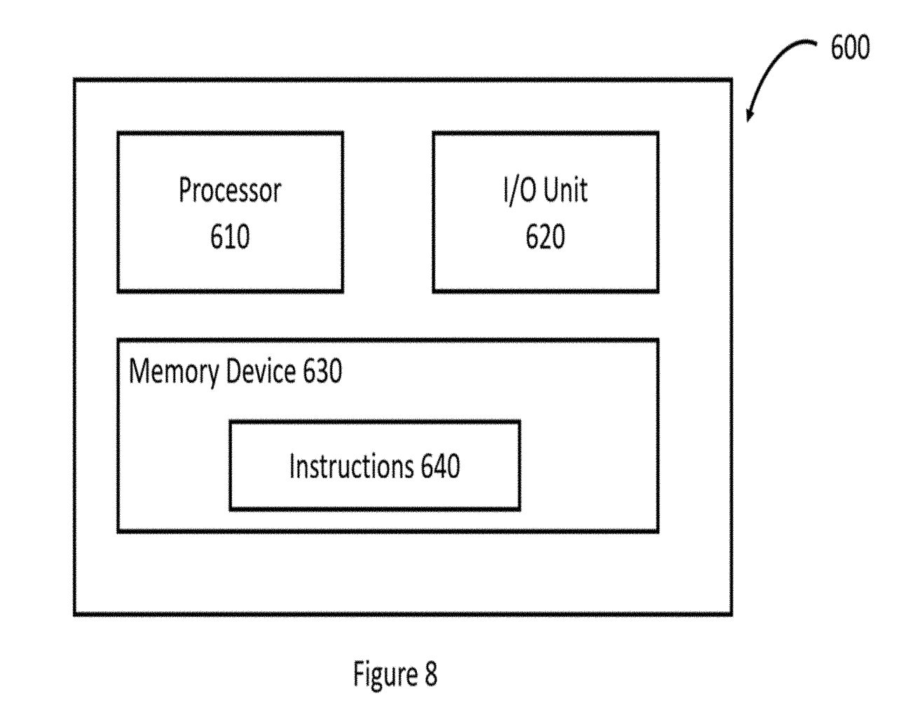

In a preferred embodiment of the invention, a health information manager component 560 is a computer with an architecture as shown in FIG. 8. Such a system may comprise one or more storage mediums or memory devices, storing computer readable instructions for retrieving health information from one or more health data providers A 140 or health data provider B 150.

The various components of the system 600, illustrated in FIG. 8, may include an assembly of hardware, software, and/or firmware, including a memory device 630, a central processing unit (“CPU”) 610, and/or a user interface using the input/output unit 620. Memory 630 may include any type of RAM or ROM embodied in a physical storage medium, such as magnetic storage including floppy disk, hard disk, or magnetic tape; semiconductor storage such as solid state disk (SSD) or flash memory; optical disc storage; or magneto-optical disc storage. A CPU may include one or more processors, such as processor 610, for processing data according to a set of programmable instructions 640 or software stored in the memory 630. The functions of each processor 610 may be provided by a single dedicated processor 610 or by a plurality of processors. Moreover, processors may include, without limitation, digital signal processor (DSP) hardware, or any other hardware capable of executing software. An optional user interface may include any type or combination of input/output devices 620, such as a display monitor, keyboard, touch screen, and/or mouse. Such a computer system 600 is an embodiment of the invention that supports the processing needs of application device 110 and health information management system 170, each having their respective instructions 640 to specify the processing as described in later sections.

In a preferred embodiment of the current invention, the health information manager 510 periodically gathers health information as configured in the database tables previously mentioned. On a periodic interval (e.g. once per day), the health information manager 510 examines each user in the user table in the configuration database 550. For a given user, the health information manager 510 examines all entries in the fetchJob table, and for each record performs the following:

1. Examine the provider table corresponding to the fetchJob record, which describes how to gather health information for this user from the indicated health data provider

2. Establish a connection to the resource on behalf of the user (e.g. call the designated script which will automatically log into the portal using the stored credentials)

3. Follow the defined navigation path, actuating links, buttons and other resources as required and as specified in the designated script, to ultimately retrieve information from the provider

4. For each item of information retrieved, perform the following:

a. Check if the item is already contained in the Health Information Database, and if it is not new, continue to the next item without storing any information.

b. Get the health information from the item. If codes are provided (ICD9, ICD10, SNOMED-CT, etc), then convert the code into readable text.

c. Create a record in the “record” table to store the retrieved health information

d. Create a “natural key” for the information, based on predefined attributes in the item. The natural key is stored as an additional column in the record table in on embodiment of the invention. A health record natural key would include a SNOMED-CT code, code system, date, provider, etc. The natural key is selected to enable de-duplication of this item from the same or other resources. The user may later choose to relate items from different resources to designate them as the same incident, condition, etc. The natural key is used for this purpose. The deduplication method may be extended to take advantage of the hierarchy of terms in the code system used, so that specific terms are known to be related to parents in the term hierarchy, and may therefore refer to the same incident, condition, etc. The user may also add notes related to this item to provide his/her personal information related to this incident/condition. The user may designate through manual indication that the record is related to an existing record, and that the two should be shown as a single incident, condition, etc.

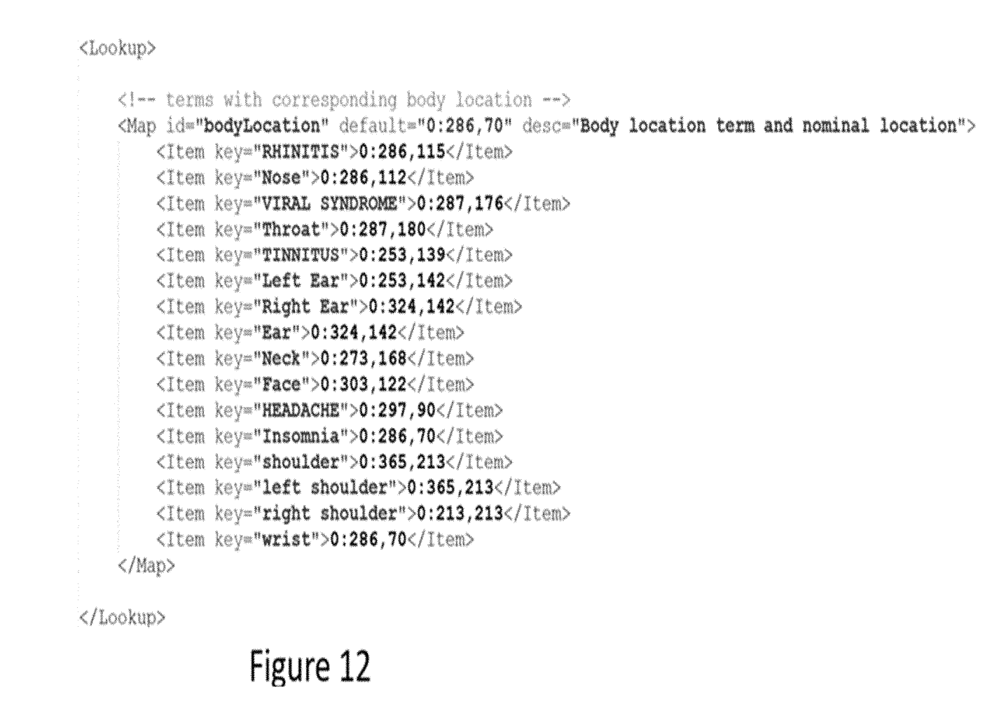

e. Look up the location on the canonical body map for the described item. In one embodiment of the current invention, each term in the item description is used to search the body map keyword reference data for a body map location. The configuration database contains a table with data similar to that represented in FIG. 12 (which is a sampling of data)., whereby a word is searched to find the location on the canonical body map. Each word in the problem description is searched until a match is found. In another embodiment of the current invention, if an ICD-9 or ICD-10 code is available, it is mapped to a SNOMED-CT code using available technologies, then the SNOMED-CT code is examined to find the corresponding “location” attribute in the SNOMED-CT data set. A lookup table is provided which maps every SNOMED-CT location attribute to a location on the canonical body map. This information is stored in a format similar to FIG. 12. The canonical body location is stored along with the record. The user is later allowed to move this to a different body map point at his/her discretion, useful when the automated means are not accurate or the user otherwise chooses a different location, in which case the new location replaces the old location in the record table.

f. Create a location for a visual text summary of the item, to be shown along with the body map. The summary text is shown in a box with color or other visual indication to designate whether the item represents a new condition, ongoing condition, or corrected condition. The location of the text is calculated to be close to the body map location, and not overlapping with other text boxes to the extent possible. A line with similar visual indications is drawn from the text box to the body map location. In order to handle large amounts of information, filters are provided to allow the user to select categories of information (e.g. dermatology, orthopedic, internal, etc). Also, a time filter can be applied to show only a time window of information.

g. Store to the health information database 560, associated with the current user, the item with its natural key, both in original item form, and in a derived form where codes and other derived information is present (e.g. body map information).

The application device 110 lets the user view the health information for themselves and for their family members, following permissions granted in the accountManager table. When a user views their information in an application (web browser or mobile device application), the following processing is performed:

1. The application presents the health information on a body map. As described earlier, each information item that is relevant to the body map, is shown in a text box with particular color or other visual indication to designate whether the item represents a new condition, ongoing condition, or corrected condition. The location of the text is calculated to be close to the body map location, and not overlapping with other text boxes to the extent possible. A line with similar visual indications is drawn from the text box to the body map location. In order to handle large numbers of information items, filters are provided to allow the user to select categories of information (e.g. dermatology, orthopedic, internal, etc). Also, a time filter can be applied to show only a time window of information. A user can scroll through their medical history for their lifetime or the lifetime a patient whose information they have been given access to.

2. Specify which items may be viewed by other users

3. Specify which items may be shared, outside the system (as when records are sent to a provider before an appointment, or when records are sent to a diagnostic system)

4. Create and edit new information records

5. Add notes to existing records

6. Allow viewing of the items in a Facebook like timeline view, in reverse chronological order

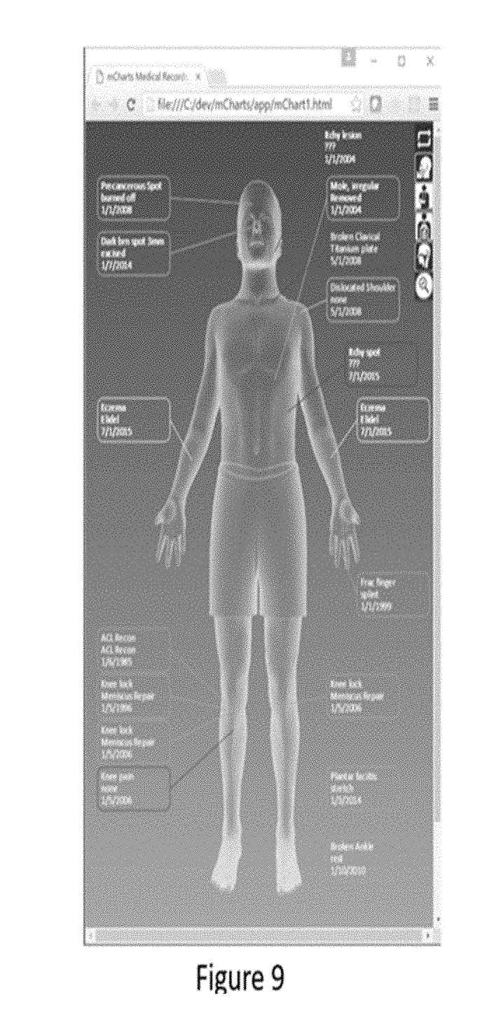

The health information manager 510 provides the ability to render the information from health data providers onto various renditions of a body map. This enables a user to view health information on a realistic, photographic picture, a wire-frame, or other visual model. This capability aids the patient and caregivers in understanding of conditions. The health information manager 510 relates conditions to body locations if appropriate. For example, a mole located 2 centimeters directly below the center of the left eye can be plotted on a body map at a point equivalent to 2 cm below the left eye center, and an appropriate annotaction can be made indicating “mole removed by nitrogen freezing”. This aids the patient and caregivers in recall and understanding of the condition and treatment, especially over time when memory tends to fade.
define a Canonical Body Map as shown in FIG. 9. This is a graphic image that is stored in the configuration database and made available to the health information manager 510 and displayed on the application device 160.
all mapping of conditions occur on the Canonical Body Map. When a textual account of a condition is stored, or a SNOMED-CT or other code is encountered representing a condition, a process is used to translate the body location to a point on the Canonical Body Map. In addition, the patient or caregiver may specify explicitly the affected point on the Canonical Body Map by pointing and clicking using a pointing device (e.g. mouse or touch-sensitive display)
Custom body shapes are supported using a photographic morphing method. One example algorith follows:
   identify key reference 3-D points on the Canonical Body Map (x,y,z)
      for example, define reference points for nose, each eye center, left, right, top, and bottom edges of mouth, tip of chin, belly button, each shoulder, each elbow, each wrist, each finger tip, low point of groin, outer edge of each hip, knee, ankle, each toe tip, etc. The precision is variable depending on the desired precision and accuracy, with more reference points producing better conversion precision at the expense of more work and processing power.
   identify corresponding reference points on the custom body shape (a,b,c). Maintain a correspondence between each reference point on the Canonical Body Map and that of the custom body shape. For example, the tip of the nose may be at location (280,110,30) on the Canonical Body Map. On the custom body map, we identify the tip of the nose using manual or automated means (image processing which can detect a specific body point). Assume this point is (1325, 1050, 330). We maintain an association table that relates the point (280,110,30) to point (1325,1050,330).
   for each point for which we need to plot onto the custom body shape (for example, a mole under the left eye as described above), start with the point on the Canonical Body Map (x,y,z). Then translate that point onto the custom body shape (a,b,c).
      Suppose the point we wish to plot is exactly on a reference point in the Canonical Body Map. Then, we simply look up the corresponding point in the custom body map and can plot on that point
      Points on the Canonical Body Map that are close to a reference point should be placed close to the corresponding reference point in the custom body shape. We create a vector that represents the distance and direction from the reference point on the Canonical Body Map. The vector is scaled as appropriate into a corresponding distance and direction from the reference point in the custom body shape. The plot point on the custom body shape is calculated as the reference point plus the scaled vector
      For points in general (that may or may not be close to a reference point), we find the closest reference point and apply the algorithm in the previous paragraph. However, since body shapes are irregular, we get a more accurate placement by selecting several of the closest reference points, applying the above algorithm to find multiple candidate points on the custom body map, then calculating the average of these points.
   Any number of custom shapes can be defined supporting the broad spectrum of body shapes found in the general population
   The algorithm to find the point on the custom body shape is intended as an initial approximate location. The user or caregiver will be allowed to later adjust the location to a more precise location visually using a pointing device or touch-sensitive display. Also, absolute accuracy is generally not needed to communicate a condition to patient or caregiver.
   The 3-D model can be expanded to incorporate other dimensions, such as age. Using age as an additional dimension, the body shape can be shown in sequence through time, thus showing the growth of child, or the change in body shape, such as while dieting and losing significant weight
   It is possible to restrict the processing to 2 dimensions insteaad of the 3 (for 3 dimensional) described here, if a flat surface map of the body is desired
   An additional dimension which selects the body map view (for example: front, left side, back, right side) can be used if flat views are desired. In this scenario, a location is constructed as (x,y,v), where ‘v’ is a flat view identifier
   Custom shapes can be created, and mapped to the Canonical Body Map. These custom shapes may include
      lifelike body shapes
      A 3-D drawing of a person
      A 3-D rendering of a person, whether drawn or derived through automated or semi-automated means
      a photograph of a person
      a sequence of photographs of a person: For example, to simulate 3 dimensional visualization, take a photograph of the individual from multiple periodic angles, identifying any key reference photographs onto which the reference points will be plotted (for example, on the front image and back image). Then, the application can show the front or back views with mapped conditions (after plotting condition locations and translating the points onto the photographic image as described above), and can show the sequence of photographs which then appear to animate the person, providing a visualization that appears to be three dimensional
      A 3-Dimensional print of the person
To increase the precision of the body map locations that we wish to plot, we define a number of Canonical Body Maps, one for each general body shape of various people across the spectrum of age, gender, weight, height, builds, length of arms, length of legs, size and shape of head, etc. We create reference locations for each of these (or derive them using the method described above, or something similar). Then an end user can select a body type that is similar to the user, to increase the precision of mapping the body location for a particular condition.

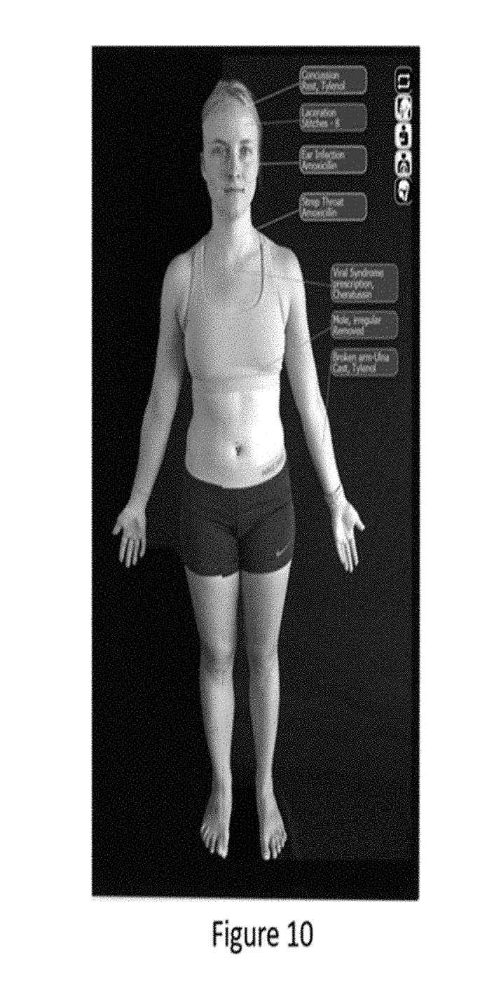

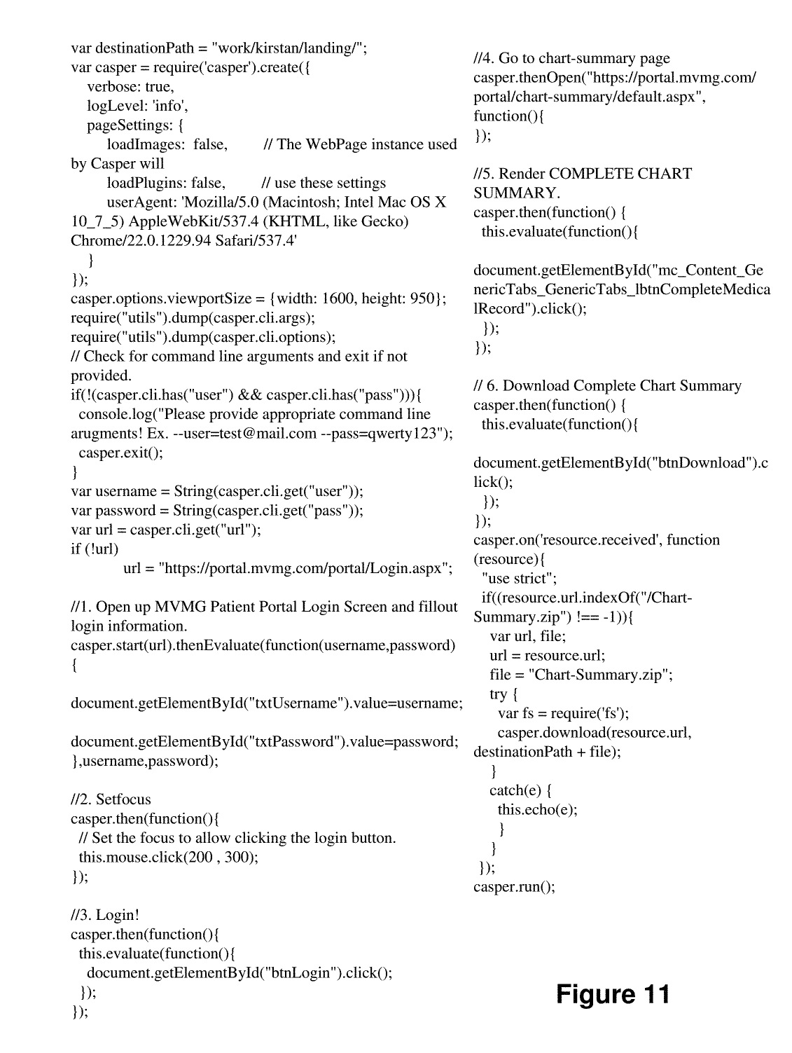

Following this process of mapping conditions to a Canonical Body Map, conditions contain references to locations on this map. Using the method described, the canonical body map location can be translated to alternate renditions of the user. FIG. 10 shows a photograph with conditions that have been plotted first on the canonical body map, then transformed to the proper locations in the image as described above. In a preferred embodiment of the invention, a user can annotate and upload a photographic image, and the conditions will be appropriately plotted on the photograph. In another embodiment, the condition can be displayed on an image while the user is a small child. As the user ages, the image can be updated to reflect growth into later stage of life, such as adolescence or adulthood. The same condition will be appropriately rendered, regardless of the shape or size of the image. In another embodiment of the preferred invention, a sequence of images of a user over time can be shown as an animated sequence, whereby the image is shown to grow frame by frame, and the conditions over time continue to be shown and accurately positioned, regardless of the size or shape of the image.

The health information manager 510 gathers information from various health data providers. FIG. 11 is one exemplary script which provides executable instructions retrieving data from a health data provider. In this embodiment, the script runs in the environment CasperJS. In other embodiments, similar scripts are developed using environments such as Selenium, GreaseMonkey, TamperMonkey, and similar tools which can automate the navigation through a patient portal or other information source, and download information. In FIG. 11, the result is downloading a Continuity of Care document, which is an XML document following HL7 standards. Once the file is downloaded, it is parsed using XPath to extract conditions, problems, procedures, etc. (each to be a type of health information item) and each is then translated into a record, the body map location is calculated as previously described, and the data is stored in the record table for later viewing. In one embodiment of the current invention, the health data provider enables access of health data through a REST interface using the FHIR standard, and the configuration information indicates which interfaces to call, supplying parameters as appropriate from configuration data, and accepting the received information into the health information manager 510, thereafter determining the canonical body map location and storing the combined information in the record table.

While the above detailed description has shown, described, and pointed out the fundamental novel features of the disclosure as applied to various implementations, it will be understood that various omissions and substitutions and changes in the form and details of the system illustrated may be made by those skilled in the art, without departing from the intent of the disclosure.

What is claimed:

1. A system for combining health information from multiple health data providers, the system comprising:
one or more storage media storing computer readable instructions; and
one or more processors configured to execute the instructions to cause the system to:
connect to one or more health data providers on behalf of a user, using the address and credentials preconfigured by a user and stored in the storage media for each health data provider
for each such configured health data provider, navigating through zero or more web pages configured in the health data provider until a download page is reached
invoking the appropriate link or command to download a health data set from the health data provider
parsing the retrieved health data set for one or more individual health information items (conditions, problems, procedures, lab results, etc) calculating the location on a canonical body map for each of the one or more health information items
storing the one or more health information items in the storage media, each along with the calculated canonical body map location
providing, at the user request, a view of a body map with the one or more health data items listed, each with a marking on the body map to visually show the body location related to the health information item.

2. The system of claim 1, wherein a second body map image is created and configured to relate to the canonical body map, and a view of the health information items is properly displayed and located on the second body map image.

3. The system of claim 2, wherein the second body map image is a photograph or sequence of photographs of the user.

4. The system of claim 1, wherein SNOMED-CT ontology is used to identify a body location of the health information item, and the list of locations is preconfigured with a location on the canonical body map.
